(19) United States
(12) Patent Application Publication
(10) Pub. No.: US 2012/0184940 A1
(43) Pub. Date: Jul. 19, 2012

(54) BIOARTIFICIAL KIDNEYS

(75) Inventors: Jackie Y. Ying, Singapore (SG); Edwin Pei Yong Chow, Singapore (SG); Jeremy C.M. Teo, Singapore (SG); Karl Schumacher, Singapore (SG); Jeremy Ming Hock Loh, Singapore (SG); Rosa Yue Qi, Singapore (SG); Qunya Ong, Singapore (SG)

(73) Assignee: Agency for Science, Technology and Research, Connexis (SG)

(21) Appl. No.: 13/499,490

(22) PCT Filed: Oct. 4, 2010
(86) PCT No.: PCT/SG2010/000377

§ 371 Date: Mar. 30, 2012

(30) Foreign Application Priority Data
Oct. 2, 2009 (SG) 200906623-4

(51) Int. Cl. A61M 1/36; A61M 1/16

(52) U.S. Cl. 604/508; 210/295

(57) ABSTRACT

The present invention generally relates to improved bioartificial kidneys (BAKs), and in certain embodiments to improved bioartificial kidneys that are portable and/or wearable by a user. In some embodiments, the BAKs may comprise an ultrafiltration unit and a reabsorption unit. The reabsorption unit may contain a reabsorption membrane having a layer of renal proximal tubule cells disposed thereon, where the renal proximal tubule cells selectively allow solutes to pass through the reabsorption membrane. In some embodiments, at least the reabsorption unit may be configured as a substantially flat-plate filtration device, which can impart advantageous properties such as improved maintenance of the renal proximal tubule cell layer, more facile monitoring of the renal proximal tubule cell layer as well as enhanced profile for wearability.

### FIELD OF INVENTION

The present invention generally relates to improved bioartificial kidneys.

### BACKGROUND

Patients with chronic kidney disease (CKD) or end stage renal failure (ESRD) experience malfunction of the nephron, the smallest functional unit of the kidney. At the onset of kidney disease, either glomerulus and/or the tubules are unable to perform their physiological function. The structure of the glomerulus determines its permselectivity, where large and/or negatively charged molecules are prevented from passing across the glomerulus unlike the small and/or positively charged ones. Such properties enable uremic substances, creatinine and urea, together with water, glucose and ions to permeate across the glomerulus as an ultrafiltrate, and at the same time retains blood cells and larger proteins within the circulatory system. The ultrafiltrate that is produced flows across the tubule of the nephron, whereby biological reabsorption of certain molecules back into the circulatory system occurs. The selective biological reabsorption of water, glucose and ions is performed by an epithelium cell layer that lines the tubules. Molecules that are not reabsorbed are removed from the body as urine. Failure of the mechanical filtration or biological reabsorption function, provided by the glomerulus or tubules respectively, would result in a plethora of clinical complications.

With prolonged life expectancy, the ratio of patients with CKD or ESRD that requires organ replacement to the number of suitable donors has increased. To enhance the survival rate of these patients, hemodialysis treatment has been employed to artificially replace the mechanical filtration function of glomerulus. Polymeric membranes with open interconnected pores, in the form of hollow fibers, are used in these dialyzers where they function as a sieving medium with carefully controlled pore sizes. This treatment is generally administered to patients 3-4 times a week for 2-4 h/treatment. Although successful, prolonged intermittent treatment may be detrimental due to hemodynamic instability as a result of large shift of solutes and fluids over a short period of time. In addition, it does not replace the lost reabsorption, metabolic and endocrine functions of the tubules. Dialyzers used for hemodialysis are therefore incomplete artificial kidney assist devices.

Recently, investigators have combined cellular functions within these mechanical devices to create bioartificial organs. Bioartificial kidneys (BAKs) containing functional kidney cells have been developed to provide the cellular functions of tubules. Within the dialyzers conventionally used for BAKs are typically thousands of hollow fiber membranes arranged in parallel. These membranes are usually fabricated from polysulfone (PS) or polyethersulfone (PES), a PS variant that is low in protein retention. In typical BAK systems, primary human kidney proximal tubule cells (HPTCs) adhere, proliferate and function on the polymeric membranes, which now also play the part of a cellular scaffold. Detailed evaluation of PS and PES membranes as substrates for renal epithelial cells has been reported in literature, and HPTCs cultivated on these substrates have produced mixed results.

### SUMMARY OF THE INVENTION

The present invention generally relates to bioartificial kidneys, and in certain embodiments to improved bioartificial kidneys that are portable and/or wearable by a user. The subject matter of the present invention involves, in some cases, interrelated products, alternative solutions to a particular problem, and/or a plurality of different uses of one or more systems and/or articles.

In one aspect, a bioartificial kidney is provided. The bioartificial kidney comprises a blood side configured to be placed in fluid communication with a blood supply and a permeate side, the blood side and the permeate side separated from each other by at least one semi-permeable membrane, wherein at least one semi-permeable membrane has a non-tubular configuration and has seeded thereon a plurality of human renal proximal tubule cells.

In some embodiments, the plurality of human renal proximal tubule cells form substantially a monolayer of cells on at least a portion of at least one semi-permeable membrane having a non-tubular configuration.

In other embodiments, the plurality of human renal proximal tubule cells form substantially a monolayer of cells on substantially the entirety of at least one side of the semi-permeable membrane of the reabsorption unit.

In yet other embodiments, the bioartificial kidney comprises a plurality of semi-permeable membranes, at least one semi-permeable membrane being essentially free of adhered cells.

In still other embodiments, the at least one semi-permeable membrane being essentially free of adhered cells is positioned in an ultrafiltration unit having an inlet in fluid communication with the blood supply, and wherein the at least one semi-permeable membrane having a non-tubular configuration and having seeded thereon a monolayer of human renal proximal tubule cells is positioned in a reabsorption unit in fluid communication with a permeate side of the ultrafiltration unit.

In yet other embodiments, the ultrafiltration unit and the reabsorption unit are contained in a single housing.

In still other embodiments, the ultrafiltration unit is contained in a first housing and the reabsorption unit is contained in a separate housing.

In another aspect, a bioartificial kidney is provided. The bioartificial kidney comprises an ultrafiltration unit comprising a blood side configured to be placed in fluid communication with a blood supply and a permeate side, the blood side and the permeate side separated from each other by a semi-permeable membrane. The bioartificial kidney further comprises a reabsorption unit in fluid communication with the permeate side of the ultrafiltration unit, the reabsorption unit comprising a retentate side and a permeate side, the retentate side and the permeate side of the reabsorption unit being separated from each other by a semi-permeable membrane, wherein the semi-permeable membrane of the reabsorption unit has a non-tubular configuration and has seeded thereon a plurality of human renal proximal tubule cells.

In some embodiments, the plurality of human renal proximal tubule cells form substantially a monolayer of cells on at least a portion of the semi-permeable membrane of the reabsorption unit.

In other embodiments, the plurality of human renal proximal tubule cells form substantially a monolayer of cells on substantially the entirety of at least one side of the semi-permeable membrane of the reabsorption unit.

In still other embodiments, the semi-permeable membrane of the reabsorption unit comprises polysulfone-Fullcure.

In yet another aspect, a bioartificial kidney is provided. The bioartificial kidney comprises an ultrafiltration unit comprising a blood side configured to be placed in fluid communication with a blood supply and a permeate side, the blood side and the permeate side separated from each other by a semi-permeable membrane. The bioartificial kidney further comprises a reabsorption unit in fluid communication with the permeate side of the ultrafiltration unit, the reabsorption unit comprising a retentate side and a permeate side, the retentate side and the permeate side of the reabsorption unit separated from each other by a semi-permeable membrane, wherein the semi-permeable membrane of the reabsorption unit comprises polysulfone-Fullcure.

In some embodiments, the semi-permeable membrane of the reabsorption unit has a non-tubular configuration.

In other embodiments, at least a portion of the semi-permeable membrane of the reabsorption unit has seeded thereon substantially a monolayer of human renal proximal tubule cells

In still other embodiments, the plurality of human renal proximal tubule cells form substantially a monolayer of cells on substantially the entirety of at least one side of the semi-permeable membrane of the reabsorption unit.

In another aspect, a method of filtering blood in an bioartificial kidney is provided. The method comprises flowing blood from a patient into a blood side of an ultrafiltration unit if the bioartificial kidney that is configured to be placed in fluid communication with the blood supply, passing at least a portion of a fluid component of the blood through a semi-permeable membrane to form a permeate on a permeate side, of the ultrafiltration unit, flowing at least a portion of the permeate into a retentate side of a reabsorption unit of the bioartificial kidney, passing at least a portion of the permeate from the ultrafiltration unit through a non-tubular semi-permeable membrane of the reabsorption unit that has seeded thereon human renal proximal tubule cells to form a reabsorbate in the retentate side of the reabsorption unit, and returning at least a portion of the reabsorbate to the patient.

In some embodiments, the human renal proximal tubule cells form substantially a monolayer on at least a portion of the semi-permeable membrane of the reabsorption unit.

In other embodiments, the human renal proximal tubule cells form substantially a monolayer of cells on substantially the entirety of at least one side of the semi-permeable membrane of the reabsorption unit.

In still other embodiments, the bioartificial kidney is capable of filtering the blood supply of the patient continuously for at least 1 day without substantial fouling.

In yet other embodiments, the ultrafiltration unit and the reabsorption unit are contained in a single housing.

In still other embodiments, the ultrafiltration unit is contained in a first housing and the reabsorption unit is contained in a separate housing.

In yet other embodiments, the semi-permeable membrane of the reabsorption unit is substantially flat.

In still other embodiments, the semi-permeable membrane of the ultrafiltration unit is substantially flat.

In yet other embodiments, the bioartificial kidney is configured to be portable.

In still other embodiments, the bioartificial kidney is configured to be wearable by a user.

In yet other embodiments, the semi-permeable membrane of the ultrafiltration unit has a molecular weight cut-off of less than 10 kDa.

In still other embodiments, the semi-permeable membrane of the ultrafiltration unit has a thickness between 50 microns and 500 microns.

In yet other embodiments, the semi-permeable membrane of the reabsorption unit has a thickness between 10 microns and 200 microns.

In still other embodiments, any of the bioartificial kidneys or methods above further comprise a membrane support layer in the reabsorption unit configured to provide support to the semi-permeable membrane of the reabsorption unit to resist applied pressure.

In yet other embodiments, any of the bioartificial kidneys or methods above further comprise a membrane support layer in the ultrafiltration unit configured to provide support to the semi-permeable membrane of the ultrafiltration unit to resist applied pressure.

In still other embodiments, the permeate side of the ultrafiltration unit contains channels having a smaller cross-sectional area than channels in the blood side of the ultrafiltration unit.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 shows an exploded view of a miniaturized flat-bed BAK comprising the ultrafiltration and reabsorption units, according to an embodiment;

FIG. 2 shows a schematic of fluid flow within the miniaturized flat-bed BAK, according to an embodiment;

FIG. 3 shows an ultrafiltration chamber containing two polycarbonate flat plates that sandwich a customized microporous polymeric membrane, according to an embodiment;

FIGS. 4A-4D show various graphs, according to an embodiment: (FIG. 4A) a graph showing ultrafiltration rates; (FIG. 4B) a graph showing albumin sieving coefficient; (FIG. 4C) a graph showing urea clearance; and (FIG. 4D) a graph showing creatinine clearance of (□) 20 wt % PSFC150, (▴) 20 wt % PSFC150-5 wt % MPC, (x) 20 wt % PSFC200-5 wt % MPS, and (♦) 10 kDa Pall Omega™ membranes, according to certain embodiments;

FIGS. 5A-5E show schematics of retentate chambers (FIGS. 5A and 5B) and various graphs illustrating device performance (FIGS. 5C-5E), according to an embodiment;

FIGS. 6A-6E show schematics of ultrafiltration units (FIGS. 6A and 6B) and various graphs illustrating device performance (FIGS. 6C-6E), according to an embodiment, according to an embodiment;

FIGS. 7A-7E show a photograph of a flat-bed BAK (FIG. 7A) and various graphs illustrating device performance (FIGS. 7B-7E), according to an embodiment;

FIG. 8 shows an exploded view of a reabsorption unit chamber, according to an embodiment, that comprises two polycarbonate flat plates that sandwich a customized microporous polymeric membrane;

FIG. 9 shows a patient wearing a wearable bioartificial kidney;

FIG. 10 is a photographic image showing immunofluorescent staining of HPTCs seeded onto 10 wt % PSFC200 membrane, according to an embodiment—immunostaining was performed after 2 weeks of cultivation, and ZO-1 (greyscale, top) as well as AQP-1 (red, bottom) were detected—nuclei were counterstained with DAPI (blue, bottom);

FIG. 11 is a graph showing measured transmembrane resistance for renal epithelial cells seeded on (▴) 10 wt % PSFC200 and (♦) PET membranes, according to an embodiment; and

FIG. 12A-12C show various graphs illustrating device performance, according to an embodiment.

### DETAILED DESCRIPTION

The present invention generally relates to bioartificial kidneys, and in certain embodiments to improved bioartificial kidneys that are portable and/or wearable by a user. In some embodiments, the BAKs may comprise an ultrafiltration unit and a reabsorption unit. In some embodiments, the ultrafiltration unit and the reabsorption unit may be contained in a single housing, which may be partitioned, in certain cases, into a first rigid walled compartment containing the ultrafiltration unit and a second rigid walled compartment containing the reabsorption unit. In certain other embodiments, the single housing, which may contain only a single rigid walled compartment containing both membrane(s) forming an ultrafiltration section (ultrafiltration unit) and membrane(s) forming a reabsorption unit. In certain embodiments, the ultrafiltration unit and the reabsorption unit may each be contained in a physically separate, independently movable housing, where the housings are connected in fluid communication with each other. The reabsorption unit generally contains a reabsorption membrane at least a portion of which having a plurality of renal proximal tubule cells disposed thereon, where the renal proximal tubule cells selectively allow solutes to pass through the reabsorption membrane. In certain embodiments, the plurality of human renal proximal tubule cells forms substantially a monolayer of cells on at least a portion of the semi-permeable membrane of the reabsorption unit, and in certain such embodiments the plurality of human renal proximal tubule cells forms substantially a monolayer of cells on substantially the entirety of at least one side of the semi-permeable membrane of the reabsorption unit, In some embodiments, the reabsorption unit may be configured as a substantially flat device (e.g. disk- or plate-like with a thickness substantially less than a width, length or diameter of the device), which can impart advantageous properties such as improved maintenance of the renal proximal tubule cell layer and more facile monitoring of the renal proximal tubule cell layer, as well as, in certain embodiments, greater portability and wearability.

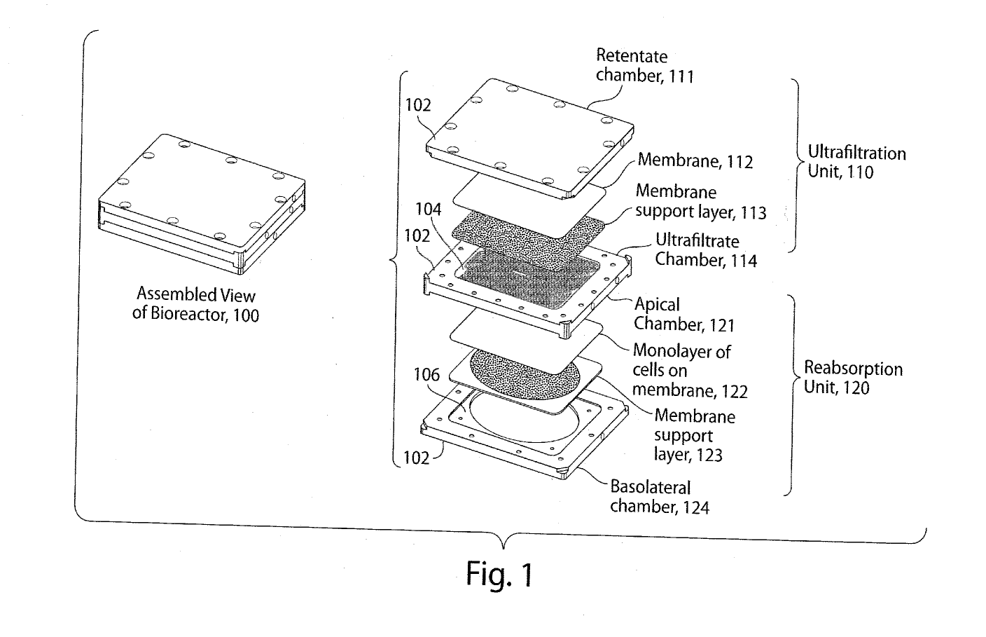

FIG. 1 shows an exploded view of an embodiment of a flat-bed BAK 100 containing a housing 102 having a first volumetric compartment 104 comprising an ultrafiltration unit 110 and a second volumetric compartment 106 comprising a reabsorption unit 120, which is described in more detail below. The compartment comprising the ultrafiltration unit contains a retentate chamber 111, an ultrafiltration membrane 112, an ultrafiltration membrane support layer 113, and a permeate chamber 114. The reabsorption unit contains an apical chamber 121, a monolayer of cells on the apical facing side of a reabsorption membrane 122, a reabsorption membrane support layer 123, and a basolateral chamber 124. Each of the two volumetric compartments comprise polymeric membranes, which are designed to replace the physiological functions of the native glomerulus and tubules. For example, the membranes used for the ultrafiltration and reabsorption units may be optimized for solute selectivity and renal epithelial cell support, respectively.

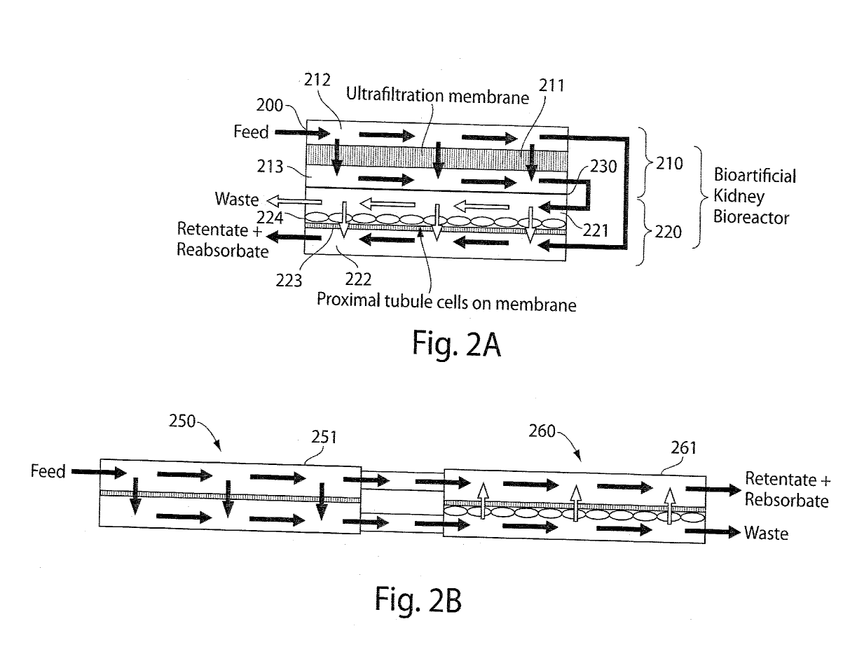

FIG. 2A shows a cross-sectional schematic depicting the operation of the embodiment of the BAK of FIG. 1. The BAK comprises an inlet 200 that is in fluid communication with the circulation system of a subject. Blood flows into the ultrafiltration unit 210 through the inlet. The ultrafiltration unit comprises an ultrafiltration membrane 211 through which fluid, but not cells, can pass. In some embodiments, a hemodialysis membrane may be used. In some embodiments, a hemofiltration membrane may be used. “Permeate” refers to the fluid that has been passed through the membrane. “Retentate” refers to the portion of the blood that does not cross the membrane. The blood flows through the inlet into the retentate chamber (i.e., the retentate side) 212, where the blood contacts the ultrafiltration membrane. Fluid from the blood passes through the ultrafiltration membrane into the permeate chamber 213 (i.e., the permeate side). The retentate and permeate then flow into the reabsorption unit 220. The reabsorption unit comprises an apical chamber (i.e., the retentate side) 221 into which the permeate from the ultrafiltration unit flows and a basolateral chamber (i.e., the permeate side) 222 into which the retentate from the ultrafiltration unit flows. The apical chamber and the basolateral chamber are separated by a membrane 223 seeded with renal epithelial cells 224. In the embodiment of FIG. 2A, the renal epithelial cells are seeded only on the side of the membrane that is in fluid communication with the apical chamber. The permeate from the ultrafiltration unit flows into the apical chamber where it contacts the renal epithelial cells. A portion of the fluid from the permeate passes through the membrane seeded with renal epithelial cells into the basolateral chamber (i.e., selected solutes are biologically reabsorbed by the membrane to elute on the basolateral side of the reabsorption unit and are mixed with the retentate from the ultrafiltration unit and recirculated). This fluid is herein referred to as the “reabsorbate.” The residual permeate containing solutes not reabsorbed by the cells flows out of the BAK and into a waste container. In some embodiments, the combined retentate and reabsorbate flows out of the BAK and back into the circulation system of a subject.

In some embodiments, the ultrafiltration unit and the reabsorption unit may be combined in a single volumetric compartment having rigid bounding walls, as opposed to the two compartment partitioned housing as illustrated in FIGS. 1 and 2A. For example, the single compartment configuration may have the rigid partition layer 230 removed forming a first, upstream region of the compartment where ultrafiltration occurs and a second, downstream region where reabsorption occurs. In some other embodiments, the ultrafiltration unit and the reabsorption unit may be contained in physically separate housings, as opposed to the two compartment partitioned housing illustrated in FIGS. 1 and 2A, that are not rigidly interconnected but are fluidically connected (see FIG. 2B). In FIG. 2B, the ultrafiltration unit 250 and the reabsorption unit 260 are contained in separate housings 251, 261.

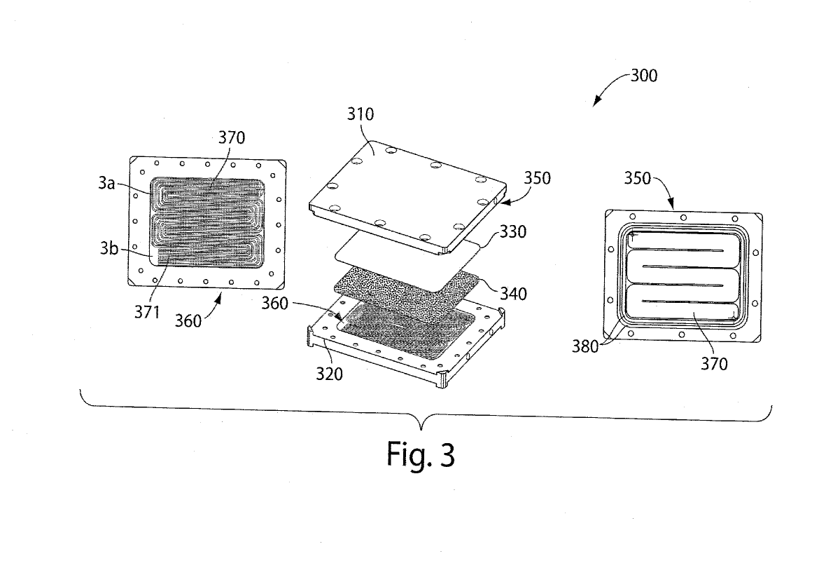

As shown in FIG. 3, in some embodiments, the ultrafiltration unit 300 may comprise two plates 310, 320 (e.g., polymer plates, such as polycarbonate) with an ultrafiltration membrane 330 and an ultrafiltration membrane support layer 340 sandwiched in between to separate the retentate chamber 350 and the permeate chamber 360 (FIG. 3). In typical embodiments, the membranes described herein (i.e., the ultrafiltration membrane, and reabsorption membrane) are semi-permeable. In some embodiments, the plates may have a serpentine flow-field layout 370 that increases the length of the flow path thereby increasing the efficiency of ultrafiltration of blood across the membrane.

As shown in FIG. 3, in some embodiments, the channels 371 in the permeate chamber may each have a smaller cross-sectional dimension transverse to the flow direction than the channels in the retentate chamber in order to increase the flow resistance of the device so as to generate a higher trans-membrane pressure (TMP). Without wishing to be bound by any theory, a higher TMP can translate to an increased ultrafiltration rate. Additionally, high TMP can cause distortion of the membrane; however, the distortion can, in some embodiments, be alleviated by the incorporation of microchannels 371 into the serpentine flow field design to act as an additional support for the membrane against flexural failure of the membrane. In some embodiments, a macroporous membrane may be placed beneath the polymeric membrane to provide additional mechanical support. In some embodiments, microchannels may be excluded from the retentate chamber so as to minimize the possibility of blood coagulation along the channel walls, which could result in the blockage of the channels and ultimately failure of the device. In some embodiments, the fluid flow resistance within the BAK may be controlled. For example, in some embodiments, the fluid flow resistance may be increased by increasing the number of microchannels in the plate adjacent to the permeate chamber. This may be desirable, for instance, for increasing the amount of permeate that passes through the ultrafiltration membrane. In certain embodiments, one or more controllable valves may be used for such purpose instead of or in addition to variation in the number of microchannels. In some embodiments, the permeate chamber channel may be sub-divided into smaller channels. These smaller channels may provide increased support to the polymeric membranes, without restricting the flow of the filtrate. In certain embodiments, a reservoir may be incorporated at the inlet to reduce the energy associated with the feed fluid, thereby preventing puncture of the membrane. In some embodiments, a double o-ring design may be used to provide an essentially leak-proof ultrafiltration unit.

In some embodiments, the retentate chamber and the permeate chamber may be configured (e.g., molded) such they may be seated together with proper alignment. For example, in some embodiments, the plates forming the two chambers may have a combination of depressions and protrusions 380 on the inside surfaces, where the depressions and protrusions align so as to align the two plates when fitted together. In some embodiments, the ultrafiltration unit may be sealed using one or more o-rings, gaskets or other sealing arrangements to make the unit essentially leak-proof.

In general, blood may be pumped along the surface of the membrane in the ultrafiltration unit by tangential flow. Solutes having a size above a threshold value generally do not pass through the pores of the ultrafiltration membrane and may be retained in the retentate chamber. Without wishing to be bound by any theory, the tangential flow can minimize fouling of the membrane by maintaining flow of the solutes in the retentate. Generally, the TMP allows sieving of smaller solutes through the pores of the membrane and into the permeate chamber (see FIG. 2A, for example).

In some embodiments, the ultrafiltration membrane is able to remove uremic substances (e.g., urea and creatinine) from blood selectively, while preventing leakage of useful proteins (e.g., albumin). In some embodiments, the pore size of the ultrafiltration membrane may be used to control the membrane selectivity. For example, in some cases, the membranes may have a total protein permeability of less than 2%, less than 1%, less than 0.5%, less than 0.2%, or less than 0.1%. In some cases, the pore size of the membrane may be chosen such that the membrane may have a predetermined molecular weight cut-off value. In some embodiments, the membrane may have a molecular weight cutoff (MWCO) that is less than ⅙ of the molecular weight of the smallest substance to be retained. For example, if albumin (MW=60 kDa) is the smallest substance that is desired to be retained, the MWCO of the membrane would be chosen to be less than about 10 kDa. In some embodiments, the MWCO of the membrane may be less than 50 kDa, less than 20 kDa, less than 10 kDa, less than 5 kDa, or less than 2 kDa.

In some embodiments, the membrane may be non-tubular in configuration. For example, in some embodiments, the membrane may be in the form of a substantially flat sheet.

In some embodiments, the ultrafiltration membrane may be fabricated from a polymeric material. For example, polymers such as polysulfone and Fullcure™ (Objet Geometries, Inc.) may be used. Additional examples of polymers that can be used to form structures described herein include but are not limited to: polyvinyl alcohol, polyvinylbutryl, polyvinylpyridyl, polyvinyl pyrrolidone, polyvinyl acetate, acrylonitrile butadiene styrene (ABS), ethylene-propylene rubbers (EPDM), EPR, chlorinated polyethylene (CPE), ethelynebisacrylamide (EBA), acrylates (e.g., alkyl acrylates, glycol acrylates, polyglycol acrylates, ethylene ethyl acrylate (EEA)), hydrogenated nitrile butadiene rubber (HNBR), natural rubber, nitrile butadiene rubber (NBR), certain fluoropolymers, silicone rubber, polyisoprene, ethylene vinyl acetate (EVA), chlorosulfonyl rubber, flourinated poly(arylene ether) (FPAE), polyether ketones, polysulfones, polyether imides, diepoxides, diisocyanates, diisothiocyanates, formaldehyde resins, amino resins, plyurethanes, unsaturated polyethers, polyglycol vinyl ethers, polyglycol divinyl ethers, poly(anhydrides), polyorthoesters, polyphosphazenes, polybutylenes, polycapralactones, polycarbonates, and protein polymers such as albumin, collagen, and polysaccharides, copolymers thereof, and monomers of such polymers. Still other examples of polymers that can be used to form structures described herein include but are not limited to: polyamines (e.g., poly(ethylene imine) and polypropylene imine (PPI)); polyamides (e.g., polyamide (Nylon), poly(e-caprolactam) (Nylon 6), poly(hexamethylene adipamide) (Nylon 66)), polyimides (e.g., polyimide, polynitrile, and poly(pyromellitimide-1,4-diphenyl ether) (Kapton)); vinyl polymers (e.g., polyacrylamide, poly(2-vinyl pyridine), polyvinylpyrrolidone), poly(methylcyanoacrylate), poly (ethylcyanoacry late), poly(butylcyanoacrylate), poly(isobutylcyanoacrylate), poly(vinyl acetate), poly (vinyl alcohol), poly(vinyl chloride), polyvinyl fluoride), poly(2-vinyl pyridine), vinyl polymer, polychlorotrifluoro ethylene, and poly(isohexylcynaoacrylate)); polyacetals; polyolefins (e.g., poly(butene-1), poly(n-pentene-2), polypropylene, polytetrafluoroethylene); polyesters (e.g., polycarbonate, polybutylene terephthalate, polyhydroxybutyrate); poly ethers (poly(ethylene oxide) (PEO), poly(propylene oxide) (PPO), poly(tetramethylene oxide) (PTMO)); vinylidene polymers (e.g., polyisobutylene, poly(methyl styrene), poly(methylmethacrylate) (PMMA), poly(vinylidene chloride), and poly(vinylidene fluoride)); polyaramides (e.g., poly(imino-1,3-phenylene iminoisophthaloyl) and poly(imino-1,4-phenylene iminoterephthaloyl)); polyheteroaromatic compounds (e.g., polybenzimidazole (PBI), polybenzobisoxazole (PBO) and polybenzobisthiazole (PBT)); polyheterocyclic compounds (e.g., polypyrrole); polyurethanes; phenolic polymers (e.g., phenol-formaldehyde); polyalkynes (e.g., poly acetylene); polydienes (e.g., 1,2-polybutadiene, cis or trans-1,4-polybutadiene); polysiloxanes (e.g., poly(dimethylsiloxane) (PDMS), poly(diethylsiloxane) (PDES), polydiphenylsiloxane (PDPS), and polymethylphenylsiloxane (PMPS)); and inorganic polymers (e.g., polyphosphazene, polyphosphonate, polysilanes, polysilazanes). Additional polymers that may be used are described in International Patent Application Serial No. PCT/US2006/035610, entitled, “Porous Polymeric Articles,” by Ying et al., filed on Sep. 12, 2006, which is incorporated herein by reference. In some embodiments, commercially available membranes, such as Pall Omega™ membranes (Pall Corporation), may be used.

In some embodiments, the membrane may be treated with or comprise one or more compositions that impart anti-fouling properties to the membrane. For example, the membrane may comprise 2-methacryloyloxyethyl phosphorylcholine, 3-methylacryloyloxy propyltrimethoxysilane, or other non-fouling compositions.

In some embodiments, the ultrafiltration membrane may be selected to yield desired performance properties. For example, decreasing the membrane thickness may allow more efficient ultrafiltration by shortening the distance that fluid must flow from the retentate chamber to the permeate chamber. The thickness of the ultrafiltration membrane may be, in some embodiments, between 50 microns and 500 microns, between 50 microns and 400 microns, between 50 microns and 300 microns, between 50 microns and 200 microns, between 100 microns and 500 microns, between 100 microns and 400 microns, or between 200 microns and 400 microns. However, decreasing the membrane thickness also may decrease the mechanical strength of the membrane. Accordingly, in some embodiments, a macroporous membrane support layer may be placed between the ultrafiltration membrane and the permeate chamber, as shown in FIGS. 1 and 3.

Any suitable biocompatible material may be used to fabricate the support layer. In some embodiments, the support layer may be macroporous relative to the ultrafiltration membrane and/or reabsorption membrane. Non-limiting examples of polymers that may be used to fabricate the support layer are provided above.

The reabsorption unit may be in fluid communication with the ultrafiltration unit. As discussed above, the permeate and retentate obtained at the end of the ultrafiltration unit can be flowed into the apical chamber and the basolateral chamber, respectively (FIG. 2A, 2B). Like the tubules of the kidney, the permeate would come into contact with the human proximal tubule cell epithelium layer, and the human proximal tubule cells would perform their biological functions in regulating the reabsorption and metabolism of important substances such as glucose, water and ions. Fluid and solutes from the permeate would then be transported across the human proximal tubule cell layer and reabsorption unit membrane into the basolateral chamber. The combined retentate and reabsorbate in the basolateral chamber may be then returned to the patient.

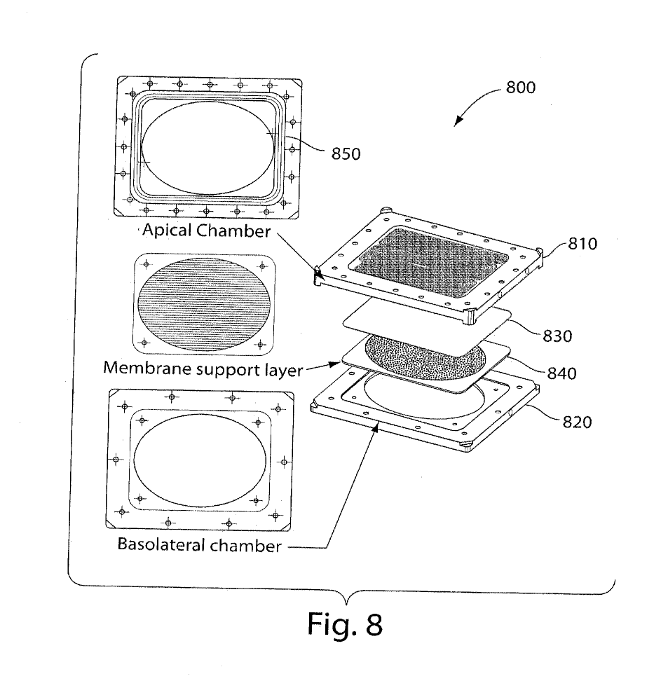

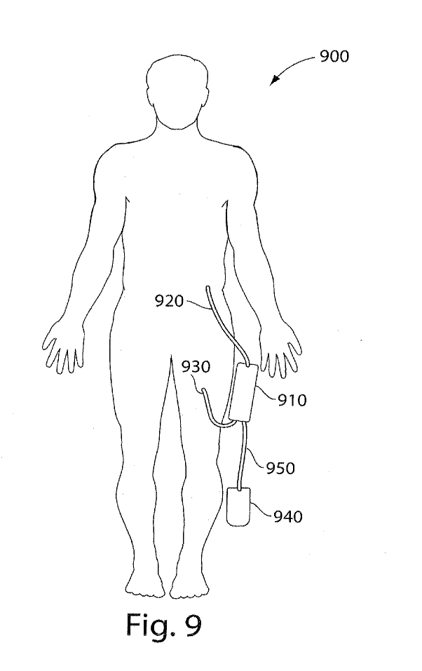

FIG. 8 shows an exploded view of one embodiment of a reabsorption unit. The unit 800 comprises an apical chamber 810 and a basolateral chamber 820. The unit also comprises a thin 100-μm reabsorption membrane 830 supported on a membrane support layer 840. The reabsorption membrane is used as a substrate for renal proximal tubule cells. A double o-ring design 850 is used to provide an essentially leakproof reabsorption unit.

In some embodiments, the reabsorption unit membrane may have a thickness of between 10 microns and 200 microns, between 50 microns and 200 microns, or between 75 microns and 150 microns. In some embodiments, the reabsorption membrane may be fabricated from a polymeric material. For example, polymers such as polysulfone and Fullcure™ (Objet Geometries, Inc.) may be used. In some embodiments, renal proximal tubule cells seeded on membranes fabricated from polysulfone and Fullcure™ may exhibit improved growth and/or morphology. Additional examples of polymers that can be used to form structures described herein include but are not limited to: polyvinyl alcohol, polyvinylbutryl, polyvinylpyridyl, polyvinyl pyrrolidone, polyvinyl acetate, acrylonitrile butadiene styrene (ABS), ethylene-propylene rubbers (EPDM), EPR, chlorinated polyethylene (CPE), ethelynebisacrylamide (EBA), acrylates (e.g., alkyl acrylates, glycol acrylates, polyglycol acrylates, ethylene ethyl acrylate (EEA)), hydrogenated nitrile butadiene rubber (HNBR), natural rubber, nitrile butadiene rubber (NBR), certain fluoropolymers, silicone rubber, polyisoprene, ethylene vinyl acetate (EVA), chlorosulfonyl rubber, flourinated poly(arylene ether) (FPAE), polyether ketones, polysulfones, polyether imides, diepoxides, diisocyanates, diisothiocyanates, formaldehyde resins, amino resins, plyurethanes, unsaturated polyethers, polyglycol vinyl ethers, polyglycol divinyl ethers, poly(anhydrides), polyorthoesters, polyphosphazenes, polybutylenes, polycapralactones, polycarbonates, and protein polymers such as albumin, collagen; and polysaccharides, copolymers thereof, and monomers of such polymers. Still other examples of polymers that can be used to form structures described herein include but are not limited to: polyamines (e.g., poly(ethylene imine) and polypropylene imine (PPI)); polyamides (e.g., polyamide (Nylon), poly(e-caprolactam) (Nylon 6), poly(hexamethylene adipamide) (Nylon 66)), polyimides (e.g., polyimide, polynitrile, and poly(pyromellitimide-1,4-diphenyl ether) (Kapton)); vinyl polymers (e.g., polyacrylamide, poly(2-vinyl pyridine), polyvinylpyrrolidone), poly(methylcyanoacrylate), poly (ethylcyanoacry late), poly(butylcyanoacrylate), poly(isobutylcyanoacrylate), poly(vinyl acetate), poly (vinyl alcohol), poly(vinyl chloride), polyvinyl fluoride), poly(2-vinyl pyridine), vinyl polymer, polychlorotrifluoro ethylene, and poly(isohexylcynaoacrylate)); polyacetals; polyolefins (e.g., poly(butene-1), poly(n-pentene-2), polypropylene, polytetrafluoroethylene); polyesters (e.g., polycarbonate, polybutylene terephthalate, polyhydroxybutyrate); poly ethers (poly(ethylene oxide) (PEO), poly(propylene oxide) (PPO), poly(tetramethylene oxide) (PTMO)); vinylidene polymers (e.g., polyisobutylene, poly(methyl styrene), poly(methylmethacrylate) (PMMA), poly(vinylidene chloride), and poly(vinylidene fluoride)); polyaramides (e.g., poly(imino-1,3-phenylene iminoisophthaloyl) and poly(imino-1,4-phenylene iminoterephthaloyl)); polyheteroaromatic compounds (e.g., polybenzimidazole (PBI), polybenzobisoxazole (PBO) and polybenzobisthiazole (PBT)); polyheterocyclic compounds (e.g., polypyrrole); polyurethanes; phenolic polymers (e.g., phenol-formaldehyde); polyalkynes (e.g., poly acetylene); polydienes (e.g., 1,2-polybutadiene, cis or trans-1,4-polybutadiene); polysiloxanes (e.g., poly(dimethylsiloxane) (PDMS), poly(diethylsiloxane) (PDES), polydiphenylsiloxane (PDPS), and polymethylphenylsiloxane (PMPS)); and inorganic polymers (e.g., polyphosphazene, polyphosphonate, polysilanes, polysilazanes). Additional polymers that may be used are described in International Patent Application Serial No. PCT/US2006/035610, entitled, “Porous Polymeric Articles,” by Ying et al., filed on Sep. 12, 2006, which is incorporated herein by reference. In some embodiments, commercially available membranes, such as Pall Omega™ membranes (Pall Corporation), may be used.

In some embodiments, a reabsorption unit membrane support layer may be included between reabsorption unit membrane and the basolateral chamber to provide mechanical support for the reabsorption unit membrane and to prevent sagging of the reabsorption unit membrane due to differential pressure across the apical chamber and the basolateral chamber.

In some embodiments, the apical chamber and basolateral chamber may be configured (e.g., molded) such they may be seated together with proper alignment in a similar fashion as described above for the retantate and permeate chambers of the ultrafiltration unit. In some embodiments, the reabsorption unit may be sealed using one or more o-rings, gaskets or other sealing arrangements to provide an essentially leak-proof seal. In some embodiments, the one or more o-rings may aid in preventing outside microbial infection of the proximal tubule cells.

In some embodiments, the membrane used in the reabsorption unit should be able to facilitate the attachment, proliferation, and support of the proximal tubule cell epithelium layer. As discussed above, certain polymers such as polysulfone and Fullcure™ may be chosen that allow improved performance of renal proximal tubule cells. In some embodiments, the reabsorption unit membrane may have molecular weight cutoff of less than 10 kDa, less than 20 kDa, less than 30 kDa, less than 40 kDa, less than 50 kDa, less than 60 kDa, or less than 80 kDa.

In some embodiments, the cell layer on the reabsorption unit membrane may comprise renal proximal tubule cells. The renal proximal tubule cells may be obtained from human subjects or other mammalian subjects. In certain embodiments, the cells form a continuous layer on the reabsorption unit membrane such that permeate cannot pass through the reabsorption unit membrane without passing through the renal proximal tubule cell layer. For example, in some embodiments, the cells form a confluent epithelium on the membrane. In certain embodiments, the paracellular spaces may be sealed by tight junctions. In some embodiments, the cells form a monolayer on the surface of the reabsorption membrane. In some embodiments, the renal proximal tubule cells may be co-cultured with other cells. For example, in certain embodiments, the renal proximal tubule cells may be co-cultured with renal cell types (e.g. distal tubule cells, collecting duct cells, podocytes and renal fibroblasts) or endothelial cells. In some embodiments, the performance of renal proximal tubule cells (e.g., the ability to reabsorb substances) may be improved in co-cultures.

In some embodiments, one or more agents can be used to promote formation and/or maintenance of renal proximal tubule cell morphology and confluence. For example, in some embodiments, bone morphogenic protein 7 (BMP-7) may be used. In some embodiments, the one or more agents may be released in controlled fashion from within the BAK. In some cases, the one or more agents may be produced within the renal tubule cells.

In some embodiments, the BAK may be configured to be portable. For example, the BAK may be a wearable device, i.e., a device worn on a user (i.e., a subject or patient). As shown in FIG. 9, a patient 900 may wear a BAK 910 that is connected to the patient's circulatory system by an inlet tube 920 and an outlet tube 930. Also worn by the patient is a waste bag 940 for collection of waste from the BAK through waste tube 950. Advantageously, a wearable BAK presents the opportunity for a subject to have continuous blood filtration over an extended period of time. For example, the BAK may be capable of substantially continuous blood filtration for a period of at least 1 hour, at least 10 hours, at least 1 day, at least 2 days, at least 4 days, at least 1 week, at least 2 weeks, at least 1 month, at least 2 months, at least 3 months, at least 6 months, or even at least 1 year. In some embodiments, the BAK is a capable of substantially continuous blood filtration without substantial fouling compromising acceptable performance. For example, in some embodiments, the channel surfaces and/or membranes in the BAK may be substantially non-fouling during operation of the BAK.

In some embodiments, the BAK may filter blood at a rate of at least 50 mL per hour, at least 100 mL per hour, at least 200 mL per hour, at least 300 mL per hour, or at least 500 mL per hour.

In some embodiments, the BAK may comprise one or more pumps for assisting fluid flow within the device. In instances wear the BAK is wearable, the pump may be battery powered, for example.

The following examples are intended to illustrate certain embodiments of the present invention, but do not exemplify the full scope of the invention.

### Example 1

This example demonstrates the performance of an embodiment of an inventive BAK.

A BAK was constructed as shown in FIG. 1. In vitro ultrafiltration studies were conducted using polysulfone/Fullcure™ (PSFC) membranes prepared with or without anti-fouling agents, such as 2-methacryloyloxyethyl phosphorylcholine (MPC) or 3-methacryloyloxy propyltrimethoxysilane (MPS). PSFC membranes were synthesized via a modified polymerization, followed by phase inversion technique (see International Patent Application Serial No. PCT/US2006/035610, entitled, “Porous Polymeric Articles,” by Ying et al., filed on Sep. 12, 2006, which is incorporated herein by reference). By varying the weight percentages of polysulfone (PS), Fullcure (FC), MPC and/or MPS, membranes with an optimum microstructure could be obtained with or without post-treatment (Table 1). These membranes had a MWCO of <30 kDa, and were hence impermeable to albumin, while providing high permeability to water and other small molecules. They had a hydraulic permeability to water (ultrafiltration rate) of ˜10-100 ml/(h·m²·mmHg), and a diffusive permeability to creatinine and urea of >0.5×10⁻⁴ cm/s.

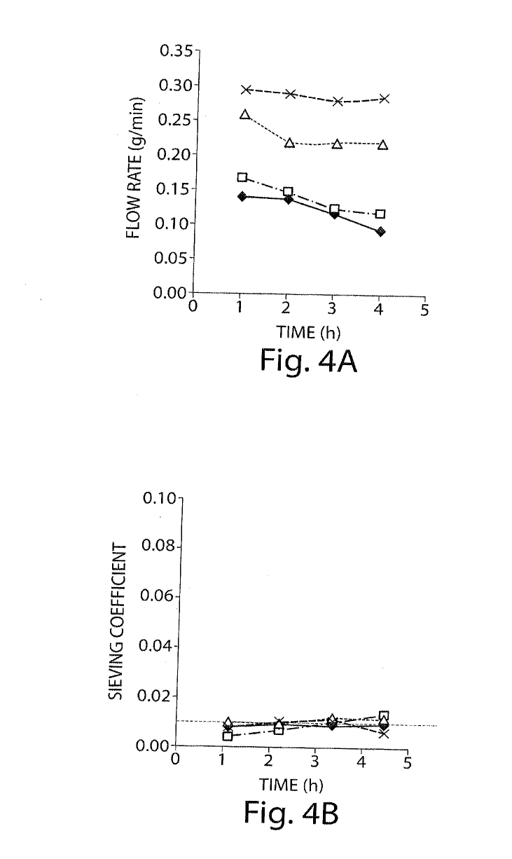

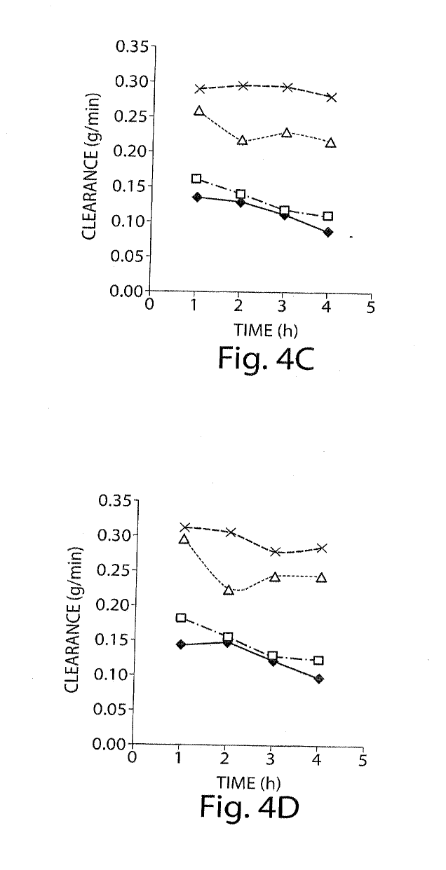

A feed solution containing 50 g/L of albumin, 0.2 g/L of urea and 0.01 g/L of creatinine, which was similar in solute concentrations as blood plasma, was pumped into the BAK at 300 ml/min for 4 h. Ultrafiltration rates, albumin sieving coefficient, urea and creatinine clearances were measured to evaluate the membrane performance (FIG. 4). PSFC membranes demonstrated excellent separation properties. They successfully filtered out low molecular weight proteins, while retaining albumin. Compared to Pall Omega™ 10 kDa membrane, PSFC membranes (especially 20 wt % PSFC200-5 wt % MPS) offered superior ultrafiltration rates, and urea and creatinine clearances. Furthermore, there was no need to stabilize the pores by post-treatment of the PSFC membranes with a liquid pore stabilizer, such as glycerol or polyethylene glycol.

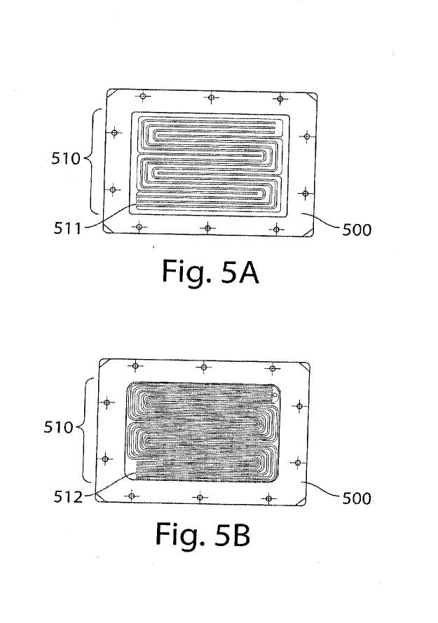

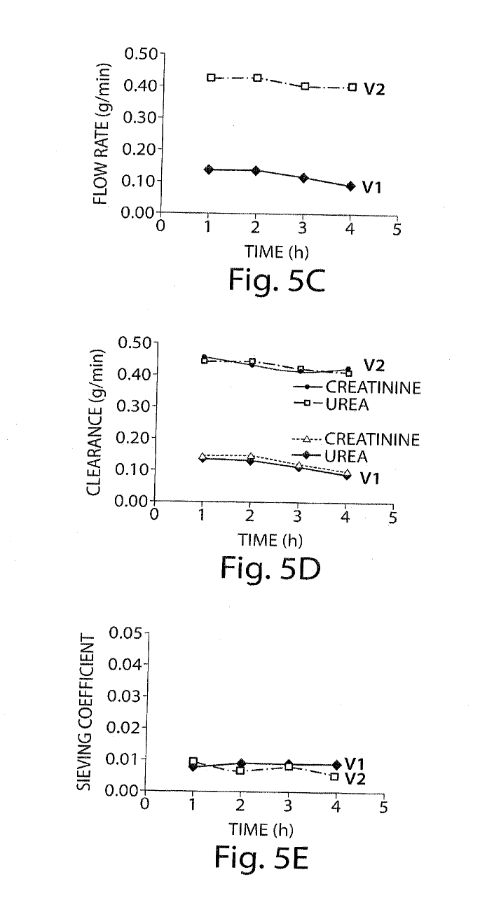

Resistance of the device could be enhanced by increasing the number of microchannels in the permeate chamber. Two ultrafiltration units with different numbers of microchannels (FIGS. 5A and 5B, showing design V1 and design V2, respectively) were tested using Pall Omega™ 10 kDa membrane. FIGS. 5A and 5B each show a permeate chamber 500 containing a serpentine channel layout 510, where the microchannels 511 in the chamber of FIG. 5A are larger than the microchannels 512 in the chamber of FIG. 5B. The device resistance increased hydraulic flow resistance, which in turn promoted ultrafiltration. At a feed flow rate of 300 ml/min, the albumin sieving values obtained were similar, but the ultrafiltration rates (FIG. 5C), and urea and creatinine clearances (FIG. 5D) were higher using a device with more microchannels (V2) as compared to a device with fewer microchannels (V1). Thus, it was plausible to increase further the ultrafiltration rate and urea and creatinine clearances without compromising albumin sieving (<0.01) (FIG. 5E) by increasing device resistance.

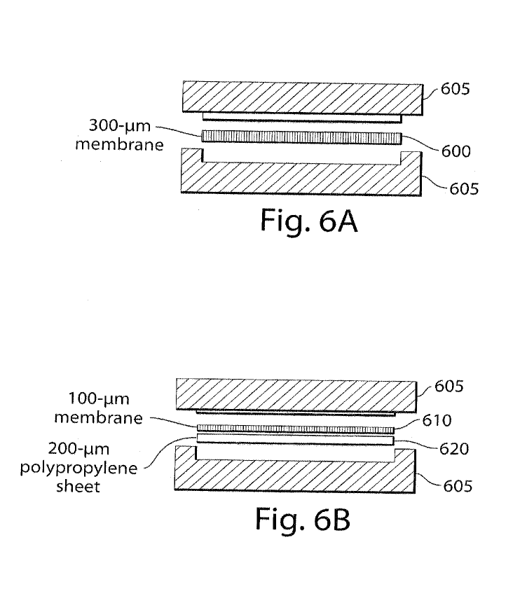

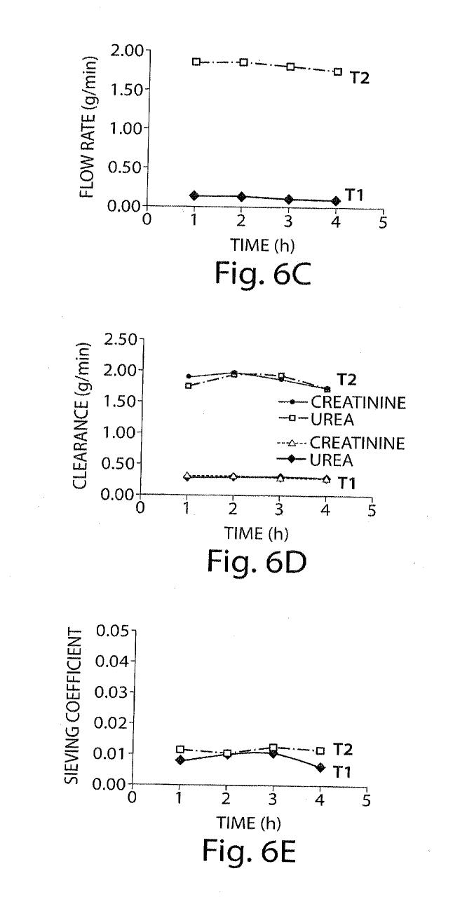

The ultrafiltration characteristics of 20 wt % PSFC200-5 wt % MPS membranes of two different thicknesses, 300 μm (FIG. 6A, 600) and 100 μm (FIG. 6B, 610), were compared. As the thinner membrane 610 has less mechanical strength, it was placed on top of a macroporous membrane support layer 620 (FIG. 6B). Cross-sections of the two setups are schematically presented in FIGS. 6A and 6B, which show the membranes within the housing 605. FIGS. 6C-6E show the ultrafiltration test results using the two different setups. The effects of membrane thickness were assessed by replacing (FIG. 6A) the 300-μm PSFC membrane (T1) with (FIG. 6B) a 100-μm PSFC membrane and a 200-μm polypropylene mesh (T2). This resulted in further increasing the ultrafiltration rate (FIG. 6C) and solute clearances (FIG. 6D). The improved efficiency of uremic substance removal was achieved at the expense of a slight increase in albumin sieving coefficient (FIG. 6E). Decrease in membrane thickness resulted in a reduction in diffusion distance and amore consistent optimal gradient for diffusion. Consequently, the fluxes of small molecules, such as water, urea and creatinine increased significantly with the use of the thinner membrane without compromising the albumin retention.

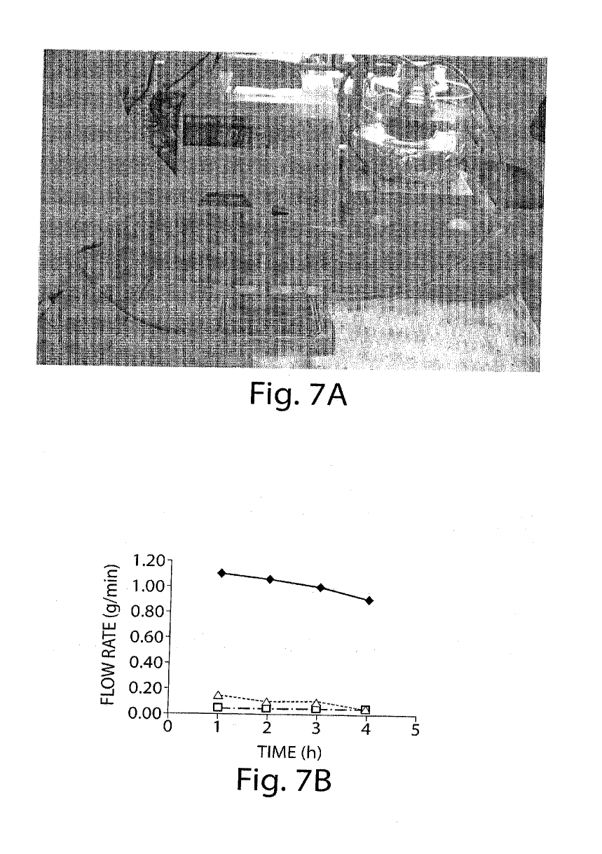

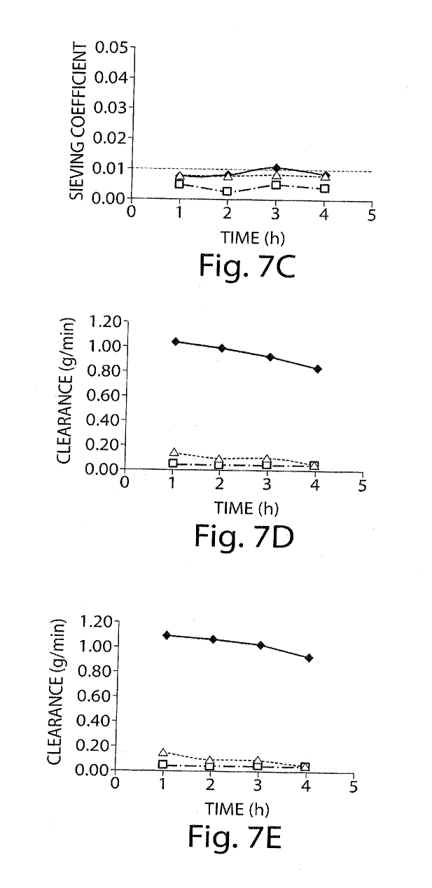

The best membrane from the in vitro ultrafiltration study (20 wt % PSFC200-5 wt % MPS) was selected for hemofiltration performance comparison with commercial membranes, such as Pall membranes of different MWCO (10 kDa and 30 kDa), in a setup 700 shown in FIG. 7A. Fresh rabbit blood was pumped at 200 ml/min through the ultrafiltration unit for 4 h to simulate clinical dialysis duration. Filtration rate, albumin sieving coefficient, and urea and creatinine clearances were measured (FIG. 7). (FIG. 7A) Rabbit blood was used as the feed solution and perfused at 300 ml/min into ultrafiltration unit of the flat-bed BAK. (FIG. 7B) Ultrafiltration rate, (FIG. 7C) albumin sieving coefficient, (FIG. 7D) urea clearance, and (FIG. 7E) creatinine clearance of (♦) 20 wt % PSFC200-5 wt % MPS, (▪) 10 kDa Pall Omega™ and (▴) 30 kDa Pall Omega™ membranes. 20 wt % PSFC200-5 wt % MPS gave the highest filtration rate and urea and creatinine clearances, while maintaining the same low albumin sieving coefficient (<0.01) as Pall Omega™ 10 kDa and Pall Omega™ 30 kDa membranes. In addition, the bioreactor system was found to be hemodynamically friendly since minimal coagulation was observed.

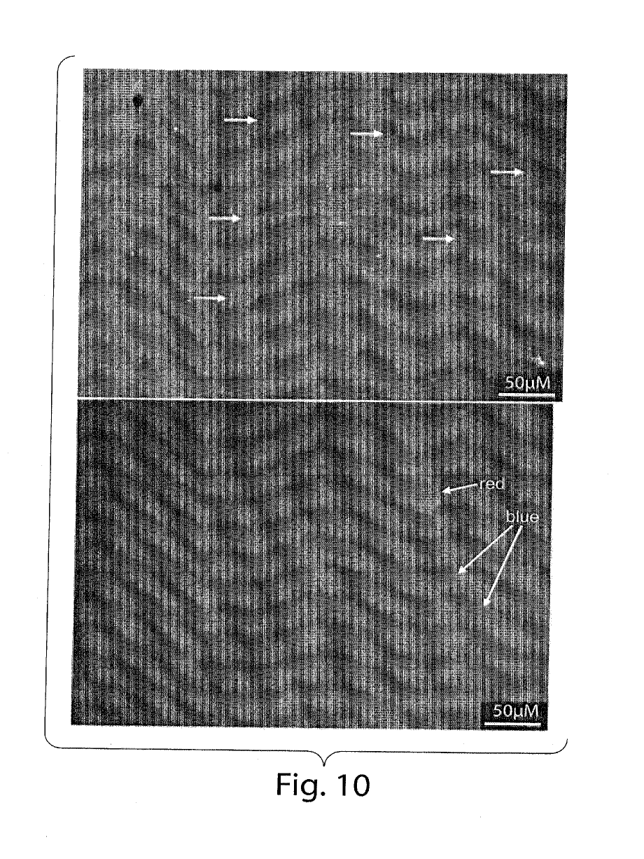

The membrane used in the reabsorption unit was configured to be able to facilitate the attachment, proliferation, and support of a well-differentiated HPTC epithelium layer. PS/polyvinylpyrrolidone (PVP) and PES/PVP membranes found in most commercial hemodialyzers were not able to perform such a function. HPTCs were seeded on PSFC membranes, and the number of live cells was determined by using the 3-(4,5-dimethylthiazol-2-yl)-5-(3-carboxymethoxyphenyl)-2-(4-sulfophenyl)-2H-tetrazolium (MTS) assay (FIG. 9). Assays were performed at (▪) day 1, (▪) week 1 and (▪) week 2 after initial seeding of 50,000 cells/cm² in triplicates to determine the average metabolic activity. Error bars indicate the standard deviations. For this study, we have optimized 10 wt % PSFC200 and found that this membrane was capable of supporting HPTCs. For BAK application, it is advantageous if HPTCs are well-differentiated and functional. The expression of aquaporin-1 (AQP-1) and zonula occluden-1 (ZO-1) were used as markers for HPTCs. AQP-1 expression is an indicator of water reabsorption functionality in the proximal tubules [S. Tang, J. C. K. Leung, C. W. K. Lam, F. M. M. Lai, T. M. Chan, K. N. Lai, Am J Kidney Diseases 2001, 38, 317]. ZO-1 expression and enrichment at lateral cell membranes would indicate formation of tight junctions sealing the epithelium. This is advantageous for selective reabsorption [B. Rothen-Rutishauser, S.D. Krämer, A. Braun, M. Günthert, H. Wunderli-Allenspach, Pharmaceutical Res 1998, 15, 964]. Immunofluorescence staining of cells seeded on 10 wt % PSFC200 membranes showed that the attached cells expressed the required ZO-1 and AQP-1 markers for well-differentiated and functional HPTCs (FIG. 10).

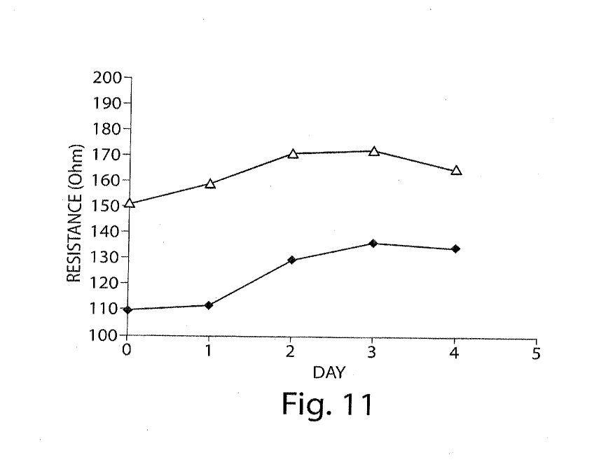

A control study was conducted to determine the cultivation duration needed to attain a renal epithelium layer on PSFC membranes. This was performed by measuring the transepithelial electric resistance across the apical and basolateral side of cell-seeded membranes using an epithelial voltohmmeter (EVOM2, World Precision Instruments, Sarasota, Fla.). Commercial polyethylene terephthalate (PET) membranes were used as a control. Resistance was measured everyday after initial cell seeding density of 50,000/cm² (FIG. 11). The results showed increasing resistance, which reached a plateau after 3 days, indicating the formation of a renal epithelium layer without leakage. Resistance for the seeded PET membranes was lower than that of the seeded PSFC membranes. This was because the PSFC membranes were thicker (average thickness=100 μm), as compared to the PET membranes (average thickness=50 μm).

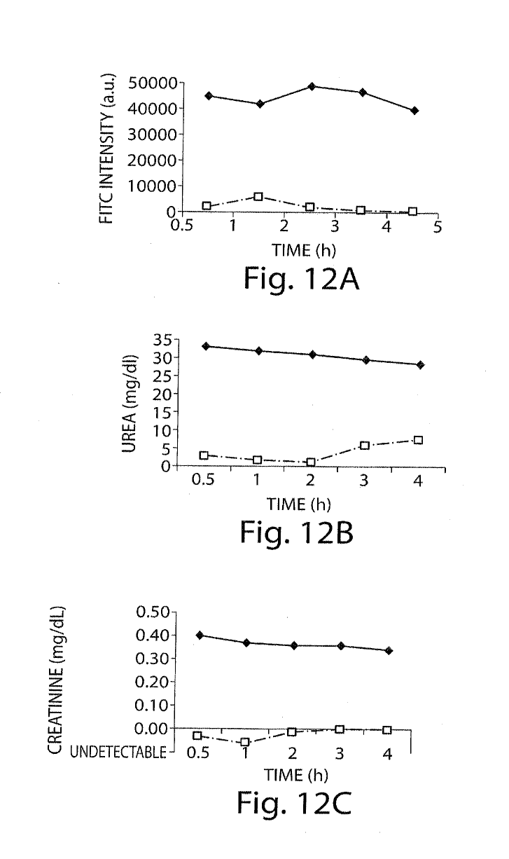

Cell-seeded PSFC membranes, cut to size, were seeded with HPTC cells and cultivated for 5 days. The cell-seeded membranes were then placed in the reabsorption unit for reabsorption studies. The apical chamber was perfused with growth factor and fetal bovine serum (FBS)-free cell culture medium that has been spiked with inulin, urea and creatinine. This condition simulated the clearance of the uremic solutes by the glomerulus into the tubules of the nephron. The basolateral chamber was perfused at a similar rate of 1 ml/min as the apical chamber, with growth factor and FBS-free cell culture medium. The low flow rate was used so that solute transport was performed entirely by the HPTC cells, and not through forced convection. The 4 h study showed that there was no leakage of inulin (FIG. 12A) or creatinine (FIG. 12C) from the apical chamber to the basolateral chamber. These results were desirable, as it indicated that a tight cell-cell interaction within the renal epithelium and that solute transport across the polymeric membrane via the cellular monolayer was biologically selective. A progressive increase and decrease in urea concentrations in the basolateral and apical chambers, respectively, was further observed (FIG. 12B). These data suggested that the functions of native tubules were mimicked in this system. Renal epithelial cell covered PSFC membranes, seeded initially at 50,000/cm², were placed into the flat-bed BAK after 3 days of cultivation. Serum-free cell culture medium, without growth factors, spiked with inulin, urea and creatinine, was perfused into the apical chamber of the reabsorption unit. The basolateral side of the reabsorption unit was perfused with serum-free cell culture medium. The concentrations of (FIG. 12A) inulin, (FIG. 12B) urea and (FIG. 12C) creatinine in the (♦) apical and (▪) basolateral chambers of the bioreactor were measured. Pump flow rates for the reabsorption unit was minimal (1 ml/min), minimizing the effects of convective movements of the solutes. Therefore, any change in solute concentrations in the apical and basolateral chamber is due to the selective solute movements induced by the cells. There are undetectable levels of creatinine in the basolateral chamber of the flat-bed BAK after 4 h of testing. This mimics the 100% rejection of the native kidney for creatinine synthesized by the body. It confirms the formation of a sheet of renal epithelium with HPTCs that have tight cell-cell interaction on the PSFC membrane. If these cells are well differentiated and functional, our reabsorption unit can replace the function of the native nephron.

TABLE 1
Composition of precursors used in the preparation
of PSFC-based ultrafiltration membranes.

| | | 20 wt % | 20 wt % |
|---|---|---|---|
| | 20 wt % | PSFC150- | PSFC200- |
| | PSFC150 | 5 wt % MPC | 5 wt % MPS |
| Polysulfone, PS [g] | 1.00 | 1.00 | 1.00 |
| Fullcure ™, FC [g] | 0.15 | 0.15 | 0.20 |
| 2-methacryloyloxyethyl | 0.00 | 0.058 | 0.00 |
| phosphorylcholine, MPC [g] | | | |
| 3-methacryloyloxy | 0.00 | 0.00 | 0.06 |
| propyltrimethoxysilane MPS [g] | | | |

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment; to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but, not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A bioartificial kidney, comprising:
a blood side configured to be placed in fluid communication with a blood supply and a permeate side, the blood side and the permeate side separated from each other by at least one semi-permeable membrane; wherein
the at least one semi-permeable membrane has a non-tubular configuration and has seeded thereon a plurality of human renal proximal tubule cells.

2. The bioartificial kidney of claim 1, wherein the plurality of human renal proximal tubule cells form substantially a monolayer of cells on at least a portion of at least one semi-permeable membrane having a non-tubular configuration.

3. The bioartificial kidney of claim 2, wherein the plurality of human renal proximal tubule cells form substantially a monolayer of cells on substantially the entirety of at least one side of the semi-permeable membrane having a non-tubular configuration.

4. The bioartificial kidney of claim 1, comprising a plurality of semi-permeable membranes, at least one semi-permeable membrane being essentially free of adhered cells.

5. The bioartificial kidney of claim 4, wherein the at least one semi-permeable membrane being essentially free of adhered cells is positioned in an ultrafiltration unit having an inlet in fluid communication with the blood supply, and wherein the at least one semi-permeable membrane having a non-tubular configuration and having seeded thereon a monolayer of human renal proximal tubule cells is positioned in a reabsorption unit in fluid communication with a permeate side of the ultrafiltration unit.

6. The bioartificial kidney of claim 5, wherein the ultrafiltration unit and the reabsorption unit are contained in a single housing.

7. The bioartificial kidney of claim 6, wherein the ultrafiltration unit is contained in a first housing and the reabsorption unit is contained in a separate housing.

8. A bioartificial kidney, comprising:
an ultrafiltration unit comprising a blood side configured to be placed in fluid communication with a blood supply and a permeate side, the blood side and the permeate side separated from each other by a semi-permeable membrane; and
a reabsorption unit in fluid communication with the permeate side of the ultrafiltration unit, the reabsorption unit comprising a retentate side and a permeate side, the retentate side and the permeate side of the reabsorption unit being separated from each other by a semi-permeable membrane; wherein
the semi-permeable membrane of the reabsorption unit has a non-tubular configuration and has seeded thereon a plurality of human renal proximal tubule cells.

9. The bioartificial kidney of claim 8, wherein the plurality of human renal proximal tubule cells form substantially a monolayer of cells on at least a portion of the semi-permeable membrane of the reabsorption unit.

10. The bioartificial kidney of claim 9, wherein the plurality of human renal proximal tubule cells form substantially a monolayer of cells on substantially the entirety of at least one side of the semi-permeable membrane of the reabsorption unit.

11. The bioartificial kidney of claim 8, wherein the semi-permeable membrane of the reabsorption unit comprises polysulfone-Fullcure™.

12. A bioartificial kidney, comprising:
an ultrafiltration unit comprising a blood side configured to be placed in fluid communication with a blood supply and a permeate side, the blood side and the permeate side separated from each other by a semi-permeable membrane; and
a reabsorption unit in fluid communication with the permeate side of the ultrafiltration unit, the reabsorption unit comprising a retentate side and a permeate side, the retentate side and the permeate side of the reabsorption unit separated from each other by a semi-permeable membrane; wherein
the semi-permeable membrane of the reabsorption unit comprises polysulfone-Fullcure™.

13. The bioartificial kidney of claim 12, wherein the semi-permeable membrane of the reabsorption unit has a non-tubular configuration.

14. The bioartificial kidney of claim 12, wherein at least a portion of the semi-permeable membrane of the reabsorption unit has seeded thereon substantially a monolayer of human renal proximal tubule cells

15. The bioartificial kidney of claim 14, wherein the plurality of human renal proximal tubule cells form substantially a monolayer of cells on substantially the entirety of at least one side of the semi-permeable membrane of the reabsorption unit.

16. A method of filtering blood in an bioartificial kidney, comprising:
flowing blood from a patient into a blood side of an ultrafiltration unit of the bioartificial kidney that is configured to be placed in fluid communication with the blood supply;
passing at least a portion of a fluid component of the blood through a semi-permeable membrane to form a permeate on a permeate side, of the ultrafiltration unit;
flowing at least a portion of the permeate into a retentate side of a reabsorption unit of the bioartificial kidney;
passing at least a portion of the permeate from the ultrafiltration unit through a non-tubular semi-permeable membrane of the reabsorption unit that has seeded thereon human renal proximal tubule cells to form a reabsorbate in the retentate side of the reabsorption unit; and
returning at least a portion of the reabsorbate to the patient.

17. The method of claim 16, wherein the human renal proximal tubule cells form substantially a monolayer on at least a portion of the semi-permeable membrane of the reabsorption unit.

18. The method of claim 17, wherein the human renal proximal tubule cells form substantially a monolayer of cells on substantially the entirety of at least one side of the semi-permeable membrane of the reabsorption unit.

19. The method of claim 16, wherein the bioartificial kidney is capable of filtering the blood supply of the patient continuously for at least 1 day without substantial fouling.

20. The bioartificial kidney of claim 8, wherein the ultrafiltration unit and the reabsorption unit are contained in a single housing.

21. The bioartificial kidney of claim 8, wherein the ultrafiltration unit is contained in a first housing and the reabsorption unit is contained in a separate housing.

22. The bioartificial kidney of claim 8, wherein the semi-permeable membrane of the reabsorption unit is substantially flat.

23. The bioartificial kidney of claim 8, wherein the semi-permeable membrane of the ultrafiltration unit is substantially flat.

24. The bioartificial kidney of claim 1, wherein the bioartificial kidney is configured to be portable.

25. The bioartificial kidney of claim 1, wherein the bioartificial kidney is configured to be wearable by a user.

26. The bioartificial kidney of claim 8, wherein the semi-permeable membrane of the ultrafiltration unit has a molecular weight cut-off of less than 10 kDa.

27. The bioartificial kidney of claim 8, wherein the semi-permeable membrane of the ultrafiltration unit has a thickness between 50 microns and 500 microns

28. The bioartificial kidney of claim 8, wherein the semi-permeable membrane of the reabsorption unit has a thickness between 10 microns and 200 microns.

29. The bioartificial kidney of claim 8, further comprising a membrane support layer in the reabsorption unit configured to provide support to the semi-permeable membrane of the reabsorption unit to resist applied pressure.

30. The bioartificial kidney of claim 8, further comprising a membrane support layer in the ultrafiltration unit configured to provide support to the semi-permeable membrane of the ultrafiltration unit to resist applied pressure.

31. The bioartificial kidney of claim 8, wherein the permeate side of the ultrafiltration unit contains channels having a smaller cross-sectional area than channels in the blood side of the ultrafiltration unit.
